SEQUENCE LISTING (8 sequences)

SEQ ID NO: 1 - DNA, 2103 bp, Mus musculus
atgctgaaacaatatattcctcttgcttttgcaccaattcctggctgcccatggctcactcagaggattcccagtagaatcgatgccaaggatggaaagacatcagttctgctctgttctcctcattctgatactattgactctggaatagaaaaagtgcctgcaagtttgactggctactctgagcttcgtgcacttgaccttgggaaaaaccaaatccaaaacatcttggaaaatggagaaatcccaggttataaagccctggaattccttagccttcatgataaccatctgcaaacacttcctaccaggtttctacatactctaccccagcttcagaagctcaacctatctatgaataagcttggaccaatcttggagcttccagaaggactctttagcacaaacttaaaagtgctagatctatcccataatcaactctgtgatgtaccccatggggctttctcccttttgtcacagctccaggagctctggttgagtggcaataacatctccagtttatccaatgaaagcctgcagggactgaggcagctgaggacactagacttaagttggaatcaaattaaagtactcaaaccaggctggctctctcatcttcctgctctgactaccttgaaccttctgggcacctacttagaaaatatcttaggcatacaacttcagggtcccaagatgctaaggcatctacaactgggttcttatccaatgctggacatatatcctccctggcccccaacactccttagcttagaaatacaagcagaatcatgtattcagtttatgattcacagtggacagccattcttattcttagagaaccttaccttagagacttccattctattactaaaaccagacaacatcacaattcattttccctccctgcgtcgcctcaccttgcgtggctacagcttcatcttctcaaccagtcaacttcagagattcttcccacaacagcttcctcttctggagcacttctttatctggtgtgaaaacagctatgcagtagacctctatctatttgggatgcccaggctacgtgtgctagagctggggtaccttaactttttctatgagtcaagtactatgaagctagagatgctattgaaggaggtacctcagttacaggtactggcattgagccacctgaatctcaggaacctctctgtgtccagctttaagagcttgcaggacctcaaactgctgctcttcaactctgaaagggcgctggagatgaacagcaacctccaggagtttattcctcagatgcctcagtacgtttacttctctgatgtcacctttacttgccagtgtgaagcttcctggctggagtcttgggctacacgggccccaaacacatttgtttatgggctggaaaaatccatttgcatagctaatgcctcagactactccaaaactctactattctctttccttgctactaattgtccacacggtactgagttttggggctttctcaccagtttcattctgctgcttctgttgattatccttcctctgattagctgtcctaaatggtcctggcttcatcacctctggacactctttcatacttgttggtggaaattatgtggacatagactcagaggccaattcaactatgatgtctttatatcctattgtgaggaggatcaagcttgggtgctggaagaactggttccagttctggagaaagcccctcctgaaggtgaaggcttgaggttgtgcctgcctgccagggactttgggattggaaatgacaggatggaatccatgattgccagcatgggcaaaagcagagccaccctctgtgtgctcacaggacaggccttagcaagtccctggtgcaatctagagttacgactggccacttaccacttggtagccaggcctgggaccactcatctcctgctgttgtttctggagccccttgataggcagaggctccatagttaccatcgcctatcccgttggctccagaaggaggactattttgatttgtcccaagggaaagtggagtggaactctttctgtgagcaactgaagagacggctcagcaaagctggacaagaaagagattaa

SEQ ID NO: 2 - protein, 700 aa, Mus musculus
MLKQYIPLAFAPIPGCPWLTQRIPSRIDAKDGKTSVLLCSPHSDTIDSGIEKVPASLTGYSELRALDLGKNQIQNILENGEIPGYKALEFLSLHDNHLQTLPTRFLHTLPQLQKLNLSMNKLGPILELPEGLFSTNLKVLDLSHNQLCDVPHGAFSLLSQLQELWLSGNNISSLSNESLQGLRQLRTLDLSWNQIKVLKPGWLSHLPALTTLNLLGTYLENILGIQLQGPKMLRHLQLGSYPMLDIYPPWPPTLLSLEIQAESCIQFMIHSGQPFLFLENLTLETSILLLKPDNITIHFPSLRRLTLRGYSFIFSTSQLQRFFPQQLPLLEHFFIWCENSYAVDLYLFGMPRLRVLELGYLNFFYESSTMKLEMLLKEVPQLQVLALSHLNLRNLSVSSFKSLQDLKLLLFNSERALEMNSNLQEFIPQMPQYVYFSDVTFTCQCEASWLESWATRAPNTFVYGLEKSICIANASDYSKTLLFSFLATNCPHGTEFWGFLTSFILLLLLIILPLISCPKWSWLHHLWTLFHTCWWKLCGHRLRGQFNYDVFISYCEEDQAWVLEELVPVLEKAPPEGEGLRLCLPARDFGIGNDRMESMIASMGKSRATLCVLTGQALASPWCNLELRLATYHLVARPGTTHLLLLFLEPLDRQRLHSYHRLSRWLQKEDYFDLSQGKVEWNSFCEQLKRRLSKAGQERD

SEQ ID NO: 3 - DNA, 2870 bp, Mus musculus
ggaccttgcaggtactctgaggtggatgagagtattggtaacccggaggcataggagtctaaagtcctctcagctctgattcctctggtgtagagatgggcaggtactggctgctgccaggtctcctcctttccctgcctctggtaactgggtggagcacttccaactgcctggtgaccgaaggctcccgactgcccctggtctcccgctatttcacattctgccgccactccaagctatcctttcttgctgcatgcctctccgtgagcaacctgacacagaccttggaagttgtacctcggactgtggaggggctctgcctcggtggtactgtgtctactctgcttccagatgctttctctgcttttcctggtctcaaggtcctggcactgagtctgcaccttacccaacttctgccaggagctctccggggtctgggacagttgcagagcctctctttttttgactctcctcttaggagatctctctttctacctcctgatgccttcagtgacctgatttccctccagagactccatatctctggcccttgcctggataagaaggcaggcatccgcctgcctcccggtctgcaatggctgggtgtcacgctcagttgcattcaggacgtgggagagctggctggtatgttcccagatctggtgcaaggttcctcctccagggtttcgtggaccctgcagaagttggatctgtcatccaaccggaagctgaagatggctagtcctgggtccctccagggtctccaggtggagattctggacctgacaagaacaccactggatgctgtgtggctgaagggcctgggacttcagaaactcgatgtcttgtatgcacagactgccacggccgagctggctgctgaggctgttgcccactttgagctgcagggcttgattgtgaaagaaagcaagataggatctatatctcaggaggctctggcttcctgccacagcctgaagaccttgggtctttcaagcactggcctaaccaagcttccaccaggcttcctgactgccatgcctaggcttcagcgactggagctgtccggaaaccaactgcagagcgccgtgctgtgcatgaatgagacgggagatgtgtcaggactcacaactctggatctgtcaggcaacaggttgcgcatcctgcctccagccgccttctcctgcttaccccacttgcgagagctgctgcttcggtacaaccagctgctttccctggagggatacctattccaggagctccagcaactagagaccttgaagctggatggaaaccccctgcttcacctgggtaagaactggttggcggctctgcctgcattgaccacccttagcttgctagatacccaaatacggatgagcccagagcctggcttctggggagcaaagaatctgcataccttgagcctgaagcttcccgctctccctgctccggcagtattgttcctgcccatgtatctgaccagcttagagcttcatatagcctcaggcacgacggagcactggacgctgtccccagagatctttccttccttggagaccttgactataagcggcgggggactgaagctgaagctggggtcccagaatgcttctggggtcttccctgctctccagaagctctccctgcttaagaacagcttggatgccttctgctcccagggtacctccaaccttttcctctggcagctccccaaacttcagtccttgagggtatggggtgctggaaacagctccagaccctgccttatcactgggctgcccagcctacgggagctgaagctggcgtcgcttcagtccataacccagccccgttcggtgcagctggaggagctggtgggtgaccttccacagctccaggccttagtgctatccagcacaggcctcaagtcactgtcggccgctgctttccagcgcctgcacagtctccaggtcttagtgctagaatacgagaaggacttgatgctgcaggacagtctgagggagtacagccctcagatgccccactatatatacattctggagtcaaacctggcctgccactgtgccaatgcgtggatggagccatgggttaagcggtccactaaaacgtacatatacataagagacaatcgcttatgtccaggacaagacaggctctctgctaggggttcccttccctcctttctctgggaccactgcccccagacgttggagctgaaactctttttggctagttctgccttggtgttcatgctaattgccttgcctctcctccaagaagccaggaactcttggatcccctacctgcaggccttgttcagggtttggctccagggtctgaggggtaagggagacaaggggaagaggttccttttcgatgtattcgtgtcccactgcaggcaagaccagggctgggtgatagaggaacttctgcctgctctggagggcttccttccagctggcctgggcctgcgcctctgtctccccgagcgtgagtttgagcctggtaaggatgtagttgataatgtggtagatagcatgttgagcagccgtaccacactctgcgtgttgagtgggcaggccctgtgtaacccccgatgccgcctggagctccgcttggccacctctctcctcctggctgccccgtcccccccagtgttgctgctagtcttcttggaacccatttctcggcaccagcttccgggttaccacagactggctcggctgcttcgaagaggagactactgtctgtggcccgaggaagaggagagaaagagtgggttctggacttggctgaggagcaggctagggtagccatagccagcactggtgtggggtggtgcatgtgaattttggggtggggttggg

SEQ ID NO: 4 - protein, 906 aa, Mus musculus
MGRYWLLPGLLLSLPLVTGWSTSNCLVTEGSRLPLVSRYFTFCRHSKLSFLAACLSVSNLTQTLEVVPRTVEGLCLGGTVSTLLPDAFSAFPGLKVLALSLHLTQLLPGALRGLGQLQSLSFFDSPLRRSLFLPPDAFSDLISLQRLHISGPCLDKKAGIRLPPGLQWLGVTLSCIQDVGELAGMFPDLVQGSSSRVSWTLQKLDLSSNRKLKMASPGSLQGLQVEILDLTRTPLDAVWLKGLGLQKLDVLYAQTATAELAAEAVAHFELQGLIVKESKIGSISQEALASCHSLKTLGLSSTGLTKLPPGFLTAMPRLQRLELSGNQLQSAVLCMNETGDVSGLTTLDLSGNRLRILPPAAFSCLPHLRELLLRYNQLLSLEGYLFQELQQLETLKLDGNPLLHLGKNWLAALPALTTLSLLDTQIRMSPEPGFWGAKNLHTLSLKLPALPAPAVLFLPMYLTSLELHIASGTTEHWTLSPEIFPSLETLTISGGGLKLKLGSQNASGVFPALQKLSLLKNSLDAFCSQGTSNLFLWQLPKLQSLRVWGAGNSSRPCLITGLPSLRELKLASLQSITQPRSVQLEELVGDLPQLQALVLSSTGLKSLSAAAFQRLHSLQVLVLEYEKDLMLQDSLREYSPQMPHYIYILESNLACHCANAWMEPWVKRSTKTYIYIRDNRLCPGQDRLSARGSLPSFLWDHCPQTLELKLFLASSALVFMLIALPLLQEARNSWIPYLQALFRVWLQGLRGKGDKGKRFLFDVFVSHCRQDQGWVIEELLPALEGFLPAGLGLRLCLPEREFEPGKDVVDNVVDSMLSSRTTLCVLSGQALCNPRCRLELRLATSLLLAAPSPPVLLLVFLEPISRHQLPGYHRLARLLRRGDYCLWPEEEERKSGFWTWLRSRLG

SEQ ID NO: 5 - DNA, 39 bp, Artificial Sequence
  primer
tcaagtctagagccaccatgctgaaacaatatattcctc

SEQ ID NO: 6 - DNA, 32 bp, Artificial Sequence
  primer
agtcatctagattaatctctttcttgtccagc

SEQ ID NO: 7 - DNA, 40 bp, Artificial Sequence
  primer
tcaagtctagagccaccatgggcaggtactggctgctgcc

SEQ ID NO: 8 - DNA, 32 bp, Artificial Sequence
  primer
aagtcctcgagctaccctagcctgctcctcag

(19) United States
(12) Patent Application Publication
(10) Pub. No.: US 2006/0121460 A1
(43) Pub. Date: Jun. 8, 2006

(54) Toll-like receptor 11

(75) Inventor: Ruslan M. Medzhitov, Branford, CT (US)

Correspondence Address: FISH & NEAVE IP GROUP;ROPES & GRAY LLP, ONE INTERNATIONAL PLACE, BOSTON, MA, 02110-2624, US

(73) Assignee: Yale University, NEW Haven, CT (US)

(21) Appl. No.: 10/511,795

(22) PCT Filed: Apr. 18, 2003
(86) PCT No.: PCT/US03/12028

§ 371 Date: Jan. 23, 2006

Related U.S. Application Data
(60) Provisional application No. 60/374,416, filed on Apr. 19, 2002.

(51) Int. Cl. C12Q 1/68; G01N 33/53; C07H 21/04; C12P 21/06; C07K 14/705; C07K 16/22; A61K 38/17

(52) U.S. Cl. 435/6; 435/7.1; 435/69.1; 435/320.1; 435/325; 514/12; 530/350; 530/388.22; 536/23.5

(57) ABSTRACT

The invention describes isolated Toll-like receptor 11 (“TLR11”) and Toll-like receptor 12 (“TLR12”) polypeptides as well as isolated variants and fragments thereof and the isolated nucleic acids encoding them. The invention also describes vectors and host cells containing nucleic acid encoding a TLR11 or a TLR12 polypeptide and methods for producing a TLR11 or a TLR12 polypeptide. Also described are methods for screening for compounds which modulate TLR11 or TLR12 activity.

### RELATED APPLICATION

This application claims the benefit of the filing date of U.S. Provisional Application No. 60/374,416, entitled “Two New Toll-like Receptor Genes: TLRL11 and TLR12”, by Ruslan Medzhitov (filed Apr. 19, 2002). The entire teachings of the referenced Provisional Application are incorporated herein by reference.

### FUNDING

Work described herein was funded, in whole or in part, by National Institutes of Health grant AI44220. The United States government has certain rights in the invention.

### BACKGROUND OF THE INVENTION

Toll-like receptors (TLRs) represent a growing family of transmembrane proteins characterized by multiple copies of leucine-rich repeats in the extracellular domain and a cytoplasmic Toll/interleukin-1 receptor (TIR) motif. TIR motifs of TLRs exhibit significant homology to the intracellular signaling domain of the type I interleukin-1 (IL-1) receptor. TLRs are evolutionarily conserved, and their congeners have been found in insects, plants, and mammals. Drosophila Toll (dToll) was the first member of the TLR family to be identified and was initially characterized as a developmental protein governing the formation of the dorsal-ventral axis in Drosophila (Belvin, M P and Anderson, K V (1996) Annu. Rev. Cell Dev. Biol. 12:393-416). However, subsequent studies revealed that dToll also plays a key role in triggering innate immune responses against fungal infection in adult flies (Anderson, K V (2000) Curr. Opin. Immunol. 12:13-19; Belvin, M P and Anderson, K V (1996) Annu. Rev. Cell Dev. Biol. 12:393-416).

To date, more than ten distinct Toll-like sequences, homologous to the highly conserved cytoplasmic domain sequence of dToll, have been identified in the largely completed Drosophila genomic sequence. In humans, nine full-length TLR sequences have also been deposited in GenBank, while six other members remain partially characterized (Anderson, K V (2000) Curr. Opin. Immunol. 12:13-19; O'Neill, L A and Greene, C (1998) J. Leukot. Biol. 63:650-7).

The Toll-like receptors (TLRs) are also thought to participate in mechanisms of innate immunity and inflammation acting as pattern recognition receptors (PRRs) for bacteria and other micro-organisms. As PRRs, TLRs recognize invariant molecular structures called pathogen-associated molecular patterns (AMPs) that are shared by many pathogens but are not expressed by hosts. TLRs are distinguished from other PRRs by their ability to recognize and discriminate between different classes of pathogens (Janeway, C A and Medzhitov, R (1999) Curr. Biol. 9:R879-R882; Anderson, K V (2000) Curr. Opin. Immunol. 12:13-19). Engagement of TLRs by pathogens leads to the activation of innate immune responses, and a major signaling target of the TLRs is activation of the transcription factor NF-κB, a key regulator of immune and inflammatory responses (Ghosh, S, et al (1998) Annu. Rev. Immunol. 16:225-260; May, M J and Ghosh, S (1998) Immunol. Today 19:80-88; and Karin, M and Ben-Neriah, Y (2000) Annu. Rev. Immunol. 18:621-663). TLR-mediated NF-κB activation is an evolutionarily conserved event that occurs in phylogenetically distinct species ranging from insects to mammals (Anderson, KV (2000) Curr. Opin. Immunol. 12:13-19; O'Neill, L A and Greene, C (1998) J. Leukot. Biol. 63:650-657). TLRs can elicit pro-inflammatory cytokine production and induce expression of cell surface co-stimulatory receptors required for activation of T-cells. Some TLRs may help to co-ordinate interactions between cells of the innate and acquired immune systems to orchestrate an integrated immune response to infection.

### SUMMARY OF THE INVENTION

The present invention relates to the discovery of two Toll-like receptors of mammalian origin, termed Toll-like receptor 11 (“TLR11”) and Toll-like receptor 12 (“TLR12”). TLR11 and TLR 12 are screening targets for the identification and development of novel pharmaceutical agents which modulate the activity of the receptors, for example, have immunomodulatory activity.

The invention relates to isolated TLR11 and TLR12 polypeptides. Polypeptide fragments or variants of a TLR11 or a TLR12 polypeptide are additional embodiments of this invention. The invention additionally relates to isolated nucleic acids (e.g., DNA, RNA) encoding a TLR11 or a TLR12 polypeptide, TLR11 or TLR12 fragments and TLR11 or TLR12 variants. The invention further relates to nucleic acids that are complementary to nucleic acid encoding a TLR11 or a TLR12 polypeptide. In certain embodiments, the invention relates to nucleic acid which hybridizes under high stringency conditions to all or a portion of nucleic acid encoding a TLR11 or a TLR12 polypeptide.

In certain aspects, the invention provides expression vectors comprising nucleic acid encoding a TLRL11 or a TLR12 polypeptide. Host cells comprising exogenous nucleic acid (e.g., DNA, RNA) encoding a TLR11 or a TLR12 polypeptide, such as host cells containing an expression vector comprising nucleic acid encoding a TLR11 or a TLR12 polypeptide, are also the subject of this invention. In another embodiment, the invention relates to a method for producing a TLR11 or a TLR12 polypeptide, such as a method of producing a TLR11 or a TLR12 polypeptide in isolated host cells containing a vector expressing a TLR11 or a TLR12 polypeptide. In certain aspects, the invention relates to an antibody that is specific for a TLR11 or a TLR12 polypeptide of the invention.

In certain embodiments, the invention provides a method of screening for compounds which modulate the activity of TLR11 and/or TLR12. Compounds (e.g., agonists or antagonists) which modulate TLR11 and/or TLR12 activity are also the subject of this invention. In another aspect, the invention provides a method of treatment for diseases affected by TLR11 and/or TLR12 activity (e.g., immune or inflammatory disorders) which includes administration of a compound which modulates TLR11 and/or TLR12 activity.

In another embodiment, the invention relates to a TLR11 or a TLR12 polypeptide, nucleic acid encoding a TLR11 or a TLR12 polypeptide, or an antibody specific for a TLR11 or a TLR12 polypeptide for use as an adjuvant or for use in the manufacture of an adjuvant or vaccine. The invention also relates to compounds which modulate TLR11 and/or TLR12 activity for use in the manufacture of a medicament for the treatment of diseases affected by TLR11 and/or TLR12 activity (e.g., immune or inflammatory disorders). TLR11 and/or TLR12 nucleic acids and the proteins encoded thereby, as well as the fragments and variants thereof, can be used as therapeutic drugs, drug targets, and for diagnostic purposes.

### BRIEF DESCRIPTION OF THE DRAWINGS

FIGS. 1A-B.

FIG. 1A shows the nucleic acid sequence encoding mouse TLR11 (SEQ ID NO: 1).

FIG. 1B shows the amino acid sequence of mouse TLR11 (SEQ ID NO: 2). Amino acids are represented by their single letter codes.

FIGS. 2A-B.

FIG. 2A shows the nucleic acid sequence encoding mouse TLR12 (SEQ ID NO: 3).

FIG. 2B shows the amino acid sequence of mouse TLR12 (SEQ ID NO: 4).

FIG. 3 is a graph depicting constitutively active TLR11 and TLR12 that activates NF-κB. Data are represented in relative light units (RLU).

### DETAILED DESCRIPTION OF THE INVENTION

The present invention provides nucleic acids and the polypeptides encoded thereby relating to two Toll-like receptors, termed Toll-like receptor 11 (“TLR11”) and Toll-like receptor 12 (“TLR12”). Described herein are isolated TLR11 and TLR 12 polypeptides, fragments and variants thereof; isolated nucleic acids (e.g., DNA, RNA) encoding TLR11 and TLR12 polypeptides, fragments and variants thereof; methods of producing TLR11 and TLR12 polypeptides; and methods in which TLR11 and TLR12 peptides are used. Such nucleic acids and polypeptides are of eukaryotic origin, such as mammalian origin (e.g. mouse, human).

In some aspects, the invention provides TLR11 and TLR12 nucleic acid sequences and proteins encoded thereby, as well as oligonucleotides derived from the nucleic acid sequences, antibodies that bind the encoded proteins, screening assays to identify agents that modulate TLR11 and/or TLR12 activity and/or biological events affected by TLR11 and/or TLR12. These compounds may be used in the treatment and/or prophylaxis of inflammatory diseases; cardiovascular diseases; systemic infections; autoimmune diseases, such as asthma; rhinitis; chronic obstructive pulmonary disease (COPD); emphysema; inflammatory bowel diseases such as ulcerative colitis and Crohn's disease; rheumatoid arthritis; osteoarthritis; psoriasis; Alzheimers disease; atherosclerosis; viral, fungal and bacterial infections, including urinary tract infections; septic shock syndrome associated with systemic infection involving gram positive and gram negative bacteria; diabetes; and Multiple Sclerosis. Agents of the invention (e.g., TLR11 polypeptides, TLR12 polypeptides, compounds which modulate TLR11 or TLR12 activity) may also be used as adjuvants to enhance or alter the immune response in vaccine therapy.

In one aspect, the invention provides an isolated nucleic acid comprising a nucleic acid which hybridizes under high stringency conditions to a nucleic acid having the sequence of SEQ ID NO: 1 or a sequence complementary thereto or having the sequence of SEQ ID NO:3 or a sequence complementary thereto. In a further embodiment, the invention is an isolated nucleic acid that is at least about 70%, 80%, 90%, 95%, 97-98%, or greater than 99% identical to a sequence corresponding to at least about 12, at least about 15, at least about 25, at least about 40, at least about 100, at least about 300, at least about 500, at least about 1000, or at least about 2100 consecutive nucleotides up to the full length of SEQ ID NO: 1 or 3, or a sequence complementary thereto. In specific embodiments, nucleic acids exhibit one of the foregoing levels of identity to SEQ ID NO: 1 or 3 and encode polypeptides that also exhibit substantially the same activity or function as TLR11 encoded by SEQ ID NO: 1 or TLR12 encoded by SEQ ID NO: 3.

Isolated nucleic acids of the present invention are relatively free from unrelated nucleic acids as well as contaminating polypeptides, nucleic acids and other cellular material that normally are associated with the nucleic acid in a cell or that are associated with the nucleic acid in a library.

In other embodiments, the invention provides expression vectors (constructs) comprising: (a) a nucleic acid which hybridizes under high stringency conditions to a sequence of SEQ ID NO: 1 or 3, or a nucleotide sequence that is at least about 70%, 80%, 90%, 95%, 97-98%, or greater than 99% identical to a sequence that is at least about 12, at least about 15, at least about 25, at least about 40, at least about 100, at least about 300, at least about 500, at least about 1000, or at least about 2100 consecutive nucleotides up to the full length of SEQ ID NO: 1 or 3, or a sequence complementary thereto, and (b) a transcriptional regulatory sequence operably linked to the nucleotide sequence. In certain embodiments, an expression vector of the present invention additionally comprises a transcriptional regulatory sequence, e.g., at least one of a transcriptional promoter or transcriptional enhancer sequence, which regulatory sequence is operably linked to the TLR11 or TLR12 sequence. In another embodiment, the nucleic acid may be included in an expression vector capable of replicating in and expressing the encoded TLR11 or TLR12 polypeptide in a prokaryotic or eukaryotic cell. In a related embodiment, the invention provides a host cell transfected with the expression vector.

Any of a wide variety of expression control sequences that control the expression of a DNA sequence when operatively linked to it may be used in these vectors to express DNA sequences encoding a TLR11 or a TLR12 polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of TLR11 or TLR12 polypeptides in cells propagated in culture, e.g., to produce proteins or polypeptides, including fusion proteins or polypeptides, for purification.

This invention also pertains to a host cell transfected with a recombinant gene comprising a coding sequence for the subject TLR11 or TLR12 polypeptides. The host cell may be any prokaryotic or eukaryotic cell. For example, a polypeptide of the present invention may be expressed in bacterial cells, such as E. coli, insect cells (e.g., using a baculovirus expression system), yeast, avian, or mammalian cells (e.g., human cells such as 293T, HeLa).

Accordingly, the present invention further pertains to methods of producing the subject TLR11 or TLR12 polypeptides. For example, a host cell transfected with an expression vector encoding a TLR11 or a TLR12 polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In one embodiment, the TLR11 or TLR12 polypeptide is a fusion protein containing a domain which facilitates its purification, such as a TLR11-GST or TLR12-GST fusion protein, TLR11-intein or TLR12-intein fusion protein, TLR11-cellulose binding domain or TLR12-cellulose binding domain fusion protein, and TLR11-polyhistidine or TLR12-polyhistidine fusion protein.

A nucleotide sequence encoding a TLR11 or a TLR12 polypeptide can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures.

A recombinant TLR11 or TLR12 nucleic acid can be produced by ligating the cloned gene, or a portion thereof, into a vector suitable for expression in either prokaryotic cells, eukaryotic cells, or both. Expression vehicles for production of recombinant TLR11 or TLR12 polypeptides include plasmids and other vectors. For instance, suitable vectors for the expression of a TLR11 or a TLR12 polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae. These vectors can replicate in E. coli due to the presence of the pBR322 ori, and in S. cerevisiae due to the replication determinant of the yeast 2 micron plasmid. In addition, drug resistance markers such as ampicillin can be used.

Certain mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. In some instances, it may be desirable to express the recombinant TLR11 or TLR12 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a TLR11 or a TLR12 polypeptide. For example, the VP6 capsid protein of rotavirus can be used as an immunologic carrier protein for portions of polypeptide, either in the monomeric form or in the form of a viral particle. The nucleic acid sequences corresponding to the portion of the TLR11 or TLR12 polypeptide to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a TLR11 or a TLR12 polypeptide and the poliovirus capsid protein can be created to enhance immunogenicity.

In yet another embodiment, the invention provides a substantially pure nucleic acid which hybridizes under high stringency conditions to a nucleic acid probe that comprises at least about 12, at least about 15, at least about 25, or at least about 40 consecutive nucleotides up to the full length of SEQ ID NO:1 or 3, or a sequence complementary thereto or up to the full length of the gene of which said sequence is a fragment. The invention also provides an antisense oligonucleotide analog which hybridizes under stringent conditions to at least 12, at least 25, or at least 50 consecutive nucleotides up to the full length of SEQ ID NO:1 or 3, or a sequence complementary thereto.

One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature. In another embodiment, the invention provides nucleic acids which hybridize under high stringency conditions of 0.5×SSC at 60° C. followed by 2 washes at 0.5×SSC at 60° C.

In a further embodiment, the invention provides a nucleic acid comprising a nucleic acid encoding the amino acid sequence of SEQ ID NO: 2 or 4, or a nucleic acid complementary thereto. In a further embodiment, the encoded amino acid sequence is at least about 70%, 80%, 90%, 95%, or 97-98%, or greater than 99% identical to a sequence corresponding to at least about 12, at least about 15, at least about 25, or at least about 40, at least about 100, at least about 200, at least about 300, at least about 400 or at least about 500 consecutive amino acid residues up to the full length of SEQ ID NO: 2 or 4.

Nucleic acids of the invention further include nucleic acids that comprise variants of SEQ ID NO:1 or 3. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NO: 1 or 3, e.g., due to the degeneracy of the genetic code. In other embodiments, variants will also include sequences that will hybridize under highly stringent conditions to a nucleotide sequence of a coding sequence designated in SEQ ID NO: 1 or 3.

Isolated nucleic acids which differ from SEQ ID NO: 1 or 3 due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. All such nucleotide variations and resulting amino acid polymorphisms are within the scope of this invention.

In another embodiment, the invention provides a probe or primer (e.g., DNA, RNA) which hybridizes under stringent conditions to at least about 12, at least about 15, at least about 25, or at least about 40 consecutive nucleotides of sense or antisense sequence selected from SEQ ID NO: 1 or 3, or a sequence complementary thereto. In certain embodiments, a probe of the present invention hybridizes to a characteristic region of SEQ ID NO: 1 or 3 and is useful to identify additional toll-like receptors. The probe may include a detachable label, such as a radioisotope, a fluorescent compound, an enzyme, or an enzyme co-factor. The invention further provides arrays of at least about 10, at least about 25, at least about 50, or at least about 100 different probes as described above attached to a solid support. Such arrays are useful to assess samples (e.g., tissues, blood, cells) for the presence of TLR11 or TLR12 nucleic acids (e.g., TLR11 mRNA, TLR12 mRNA).

Optionally, a TLR11 or a TLR12 nucleic acid of the invention will genetically complement a partial or complete TLR11 or TLR12 loss of function phenotype in a cell. For example, a TLR11 or a TLR12 nucleic acid of the invention may be expressed in a cell in which endogenous TLR11 or TLR12 has been reduced by RNAi, and the introduced TLR11 or TLR12 nucleic acid will mitigate a phenotype resulting from the RNAi. The term “RNA interference” or “RNAi” refers to any method by which expression of a gene or gene product is decreased by introducing into a target cell one or more double-stranded RNAs which are homologous to the gene of interest (particularly to the messenger RNA of the gene of interest).

Another aspect of the invention relates to TLR11 nucleic acids that are used for antisense, RNAI or ribozymes. As used herein, nucleic acid therapy refers to administration or in situ generation of a nucleic acid or a derivative thereof which specifically hybridizes (e.g., binds) under cellular conditions with the cellular mRNA and/or genomic DNA encoding one of the subject TLR11 or TLR12 polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

A nucleic acid therapy construct of the present invention can be delivered, for example, as an expression plasmid which, when transcribed in the cell, produces RNA which is complementary to at least a unique portion of the cellular mRNA which encodes a TLR11 or a TLR12 polypeptide. Alternatively, the construct is an oligonucleotide which is generated ex vivo and which, when introduced into the cell causes inhibition of expression by hybridizing with the mRNA and/or genomic sequences encoding a TLR11 or a TLR12 polypeptide. Such oligonucleotide probes are optionally modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668. Nucleic acid constructs of the invention are useful in therapeutic, diagnostic, and research contexts.

In addition to use in therapy, the oligomers of the invention may be used as diagnostic reagents to detect the presence or absence of the TLR11 or TLR12 DNA or RNA sequences to which they specifically bind, such as for determining the level of expression of a gene of the invention or for determining whether a gene of the invention contains a genetic lesion.

In another aspect, the invention provides polypeptides. In one embodiment, the invention pertains to a polypeptide encoded by a nucleic acid which hybridizes under stringent conditions to a nucleic acid of SEQ ID NO: 1 or 3, a sequence complementary thereto, or a fragment encoding an amino acid sequence comprising at least about 25, or at least about 40 amino acid residues thereof.

In another embodiment, the TLR11 or TLR12 polypeptide comprises a sequence that is identical with or homologous to SEQ ID NO: 2 or 4. For instance, a TLR11 or a TLR12 polypeptide preferably has an amino acid sequence at least 70% identical to a polypeptide represented by SEQ ID NO: 2 or 4 or an amino acid sequence that is 80%, 90% or 95% identical thereto. The TLR11 or TLR12 polypeptide can be full length, such as the polypeptide represented by the amino acid sequence in SEQ ID NO: 2 or 4 or it can comprise a fragment of, for instance, at least 5, 10, 20, 50, 100, 150, 200, 250, 300, 400 or 500 or more amino acid residues in length.

In another embodiment, the invention features a purified or recombinant polypeptide fragment of a TLR11 or a TLR12 polypeptide, which polypeptide has the ability to modulate, e.g., stimulate or antagonize, an activity of a wild-type TLR11 or TLR12 protein. In one embodiment, the polypeptide fragment comprises a sequence identical or homologous to the amino acid sequence designated in SEQ ID NO: 2 or 4.

Moreover, as described below, the TLR11 or TLR12 polypeptide can be either an agonist or alternatively, an antagonist of a biological activity of a naturally occurring form of the protein, e.g., the polypeptide is able to modulate the intrinsic biological activity of a TLR11 or a TLR12 protein or a TLR11 or a TLR12 complex, such as activation of NF-κB.

The present invention also relates to chimeric molecules, such as fusion proteins, that comprise all or a portion of a TLR11 or a TLR12 polypeptide and a second polypeptide that is heterologous (not a TLR11 or a TLR12 polypeptide), such as the extracellular domain of a CD4 receptor or an epitope tag, such as a Flag or myc epitope tag.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

The present invention also makes available isolated and/or purified forms of the subject TLR11 and TLR12 polypeptides, which are isolated from, or otherwise substantially free of, other intracellular proteins which might normally be associated with the protein or a particular complex including the protein. TLR11 and TLR12 polypeptides which are recombinantly produced (e.g., by recombinant DNA methods) or chemically synthesized are also the subject of this invention.

Optionally, a TLR11 or a TLR12 polypeptide of the invention will function in place of an endogenous TLR11 or TLR12 polypeptide, respectively, for example by mitigating a partial or complete TLR11 or TLR12 loss of function phenotype in a cell.

Variants and fragments of a TLR11 or a TLR12 polypeptide may have enhanced activity or constitutive activity, or, alternatively, act to prevent TLR11 or TLR12 polypeptides from performing one or more functions. For example, a truncated form lacking one or more domains may have a dominant negative effect.

Another aspect of the invention relates to polypeptides derived from a full-length TLR11 or TLR12 polypeptide. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, the subject protein can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, or more generally of a TLR11 or a TLR12 complex, such as by microinjection assays.

It is also possible to modify the structure of the subject TLR11 or TLR12 polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the TLR11 and TLR12 polypeptides described in more detail herein. Such modified polypeptides include peptide mimetics. Peptide mimetics include chemically modified peptides and peptide-like molecules containing non-naturally occurring amino acids. Modified polypeptides can also be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Whether a change in the amino acid sequence of a polypeptide results in a functional homolog can be readily determined by assessing the ability of the variant polypeptide to produce a response in cells in a fashion similar to the wild-type protein. For instance, such variant forms of a TLR11 or a TLR12 polypeptide can be assessed, e.g., for their ability to activate NF-κB; e.g., to stimulate the production of cytokines; e.g., to bind to another polypeptide such as for example, another TLR11 or TLR12 polypeptide or another protein involved in immunomodulatory activity. Polypeptides in which more than one replacement has taken place can readily be tested in the same manner.

This invention further contemplates a method of generating sets of combinatorial mutants of the subject TLR11 or TLR12 polypeptides, as well as truncation mutants, and is especially useful for identifying potential variant sequences (e.g., homologs) that are functional in binding to a TLR11 or a TLR12 polypeptide. The purpose of screening such combinatorial libraries is to generate, for example, TLR11 or TLR12 homologs which can act as either agonists or antagonists, or alternatively, which possess novel activities all together. Combinatorially-derived homologs can be generated which have a selective potency relative to a naturally occurring TLR11 or TLR12 polypeptide. Such proteins, when expressed from recombinant DNA constructs, can be used in gene therapy protocols.

Yet another aspect of the present invention concerns an immunogen which comprises a TLR11 or a TLR12 polypeptide capable of eliciting an immune response specific for the TLR11 or TLR12 polypeptide; e.g., a humoral response, an antibody response; or a cellular response. In certain embodiments, the immunogen comprises an antigenic determinant, e.g., a unique determinant, from a protein represented by SEQ ID NO: 2 or 4.

Another aspect of the invention pertains to an antibody specifically reactive with a TLR11 or a TLR12 polypeptide. For example, by using immunogens derived from a TLR11 or a TLR12 polypeptide, e.g., based on the cDNA sequences, anti-protein/anti-peptide antisera or monoclonal antibodies can be made by standard protocols. A mammal, such as a mouse, a hamster or rabbit can be immunized with an immunogenic form of the peptide (e.g., a TLR11 or a TLR12 polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a TLR11 or a TLR12 polypeptide can be administered in the presence of adjuvant. The progress of immunization can be monitored by detection of antibody titers in plasma or serum. Standard ELISA or other immunoassays can be used with the immunogen as antigen to assess the levels of antibodies. In one embodiment, the subject antibodies are immunospecific for antigenic determinants of a TLR11 or a TLR12 polypeptide of a mammal, e.g., antigenic determinants of a protein set forth in SEQ ID NO: 2 or 4.

In another embodiment, the antibodies are immunoreactive with one or more proteins having an amino acid sequence that is at least 70% identical, at least 80% identical to an amino acid sequence as set forth in SEQ ID NO: 2 or 4. In other embodiments, an antibody is immunoreactive with one or more proteins having an amino acid sequence that is 75%, 80%, 85%, 90%, 95%, 98%, 99% or identical to an amino acid sequence as set forth in SEQ ID NO: 2 or 4.

Following immunization of an animal with an antigenic preparation of a TLR11 or a TLR12 polypeptide, anti-TLR11 or anti-TLR12 antisera can be obtained and, if desired, polyclonal anti-TLR11 or anti-TLR12 antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian TLR11 or TLR12 polypeptide of the present invention and monoclonal antibodies isolated from a culture comprising such hybridoma cells. In one embodiment, anti-mouse TLR11 or anti-mouse TLR12 antibodies specifically react with the protein encoded by a nucleic acid having SEQ ID NO: 1 or 3, respectively.

The term antibody as used herein is intended to include fragments thereof which are also specifically reactive with one of the subject TLR11 or TLR12 polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a TLR11 or a TLR12 polypeptide conferred by at least one CDR region of the antibody. In certain embodiments, the antibody further comprises a label attached thereto and able to be detected, (e.g., the label can be a radioisotope, fluorescent compound, enzyme or enzyme co-factor).

An application of anti-TLR11 or anti-TLR12 antibodies of the present invention is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. For instance, gt11 will produce fusion proteins whose amino termini consist of β-galactosidase amino acid sequences and whose carboxy termini consist of a foreign polypeptide. Antigenic epitopes of a TLR11 or a TLR12 polypeptide, e.g., other orthologs of a particular protein or other paralogs from the same species, can then be detected with antibodies, as, for example, reacting nitrocellulose filters lifted from infected plates with the appropriate anti-TLR11 or anti-TLR12 antibodies. Positive phage detected by this assay can then be isolated from the infected plate. Thus, the presence of TLR11 or TLR12 homologs can be detected and cloned from other animals, including humans.

In certain embodiments, the present invention provides assays for identifying therapeutic agents which either interfere with or promote TLR11 and/or TLR12 function. In certain embodiments, agents of the invention specifically modulate TLR11 activity. In another embodiment, agents of the invention specifically modulate TLR12 activity. In certain embodiments, agents of the invention modulate the activity of TLR11 and/or TLR12 and may be used to treat certain diseases related to an inflammatory disorder, an autoimmune disease, a cardiovascular disorder, or a systemic infection that is responsive to Toll-like receptor modulation. In certain embodiments, agents of the invention modulate the activity of TLR11 and/or TLR12 and may be used to treat a viral, fungal or bacterial infection, including urinary tract infections; asthma; rhinitis; chronic obstructive pulmonary disease (COPD); emphysema; an inflammatory bowel disease such as ulcerative colitis or Crohn's disease; rheumatoid arthritis; osteoarthritis; psoriasis; Alzheimers disease; atherosclerosis, Multiple Sclerosis; diabetes; and septic shock syndrome associated with systemic infection involving gram positive or grain negative bacteria. In certain embodiments, the invention provides assays to identify, optimize or otherwise assess agents that increase or decrease the activity of a TLR11 polypeptide, a TLR12 polypeptide or both a TLR11 and a TLR12 polypeptide.

In certain embodiments, an assay comprises screening for activation of NF-κB. For example, mammalian cells such as 293T cells transfected with an NF-κB luciferase reporter construct and expressing a constitutively active TLR11 or TLR12 polypeptide or TLR11 or TLR12 fusion protein (e.g., the cytoplasmic domain of TLR11 or TLR12 fused to the extracellular domain of a CD4 receptor) are assayed for NF-κB activation. For instance, activation of NF-κB by constitutively active TLR11 or TLR12 is measured by NF-κB induced luciferase activity which is measured by means of a luminometer.

Alternatively, in certain embodiments, an assay comprises screening for activation of NF-κB by TLR11 or TLR12 polypeptides activated by means of an agent such as an endogenous ligand or a therapeutic compound. For example, mammalian cells such as 293T cells are transfected with an NF-κB luciferase reporter construct and express a TLR11 or a TLR12 polypeptide. The TLR11 or TLR12 polypeptide is contacted with an agent which activates TLR11 or TLR12. TLR11 or TLR12 activation by the agent is measured by the activation of NF-κB, which activity is measured by luciferase activity by means of a luminometer.

Alternatively, an assay comprises detecting the production of cytokines. For example, mammalian cells such as RAW 264.7 macrophages expressing a constitutively active TLR11 or TLR12 polypeptide or TLR11 or TLR12 fusion protein (e.g., the cytoplasmic domain of TLR11 or TLR12 fused to the extracellular domain of a CD4 receptor) are tested for production of a cytokine at the cell surface of the cells by immunostaining for TNF-α followed by flow cytometry.

An assay as described above may be used in a screening assay to identify agents that modulate an immunomodulatory activity of a TLR11 and/or TLR12 polypeptide. A screening assay will generally involve adding a test agent to one of the above assays, or any other assay designed to assess an immunomodulatory-related activity of a TLR11 or a TLR12 polypeptide. The parameters detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be preferred.

In a screening assay, the effect of a test agent may be assessed by, for example, assessing the effect of the test agent on kinetics, steady-state and/or endpoint of the reaction.

Certain embodiments of the invention relate to assays for identifying agents that bind to a TLR11 or a TLR12 polypeptide, optionally a particular domain of TLR11 or TLR12 such as an extracellular domain (e.g., a leucine rich repeat domain) or an intracellular domain such as a TIR domain. In certain embodiments, the invention relates to assays for identifying agents that bind to both a TLR11 and a TLR12 polypeptide. A wide variety of assays may be used for this purpose, including labeled in vitro protein-protein binding assays, electrophoretic mobility shift assays, and immunoassays for protein binding. The purified protein may also be used for determination of three-dimensional crystal structure, which can be used for modeling intermolecular interactions and design of test agents. In one embodiment, an assay detects agents which inhibit the activation of one or more subject TLR11 and/or TLR12 polypeptides. In another embodiment, the assay detects agents which modulate the intrinsic biological activity of a TLR11 and/or a TLR12 polypeptide, such as activation of NF-κB or stimulation of the production of cytokines.

In additional embodiments of the invention, assay formats include those which approximate such conditions as formation of protein complexes, enzymatic activity, and TLR11 or TLR12 immunomodulatory activity, e.g., purified proteins or cell lysates, as well as cell-based assays which utilize intact cells. Simple binding assays can also be used to detect agents which bind to TLR11 and/or TLR12. Such binding assays may also identify agents that act by disrupting the interaction between a TLR11 or a TLR12 polypeptide and a TLR11 or a TLR12 interacting protein, respectively or the binding of a TLR11 or a TLR12 polypeptide or complex to a substrate. Agents to be tested can be produced, for example, by bacteria, yeast or other organisms (e.g., natural products), produced chemically (e.g., small molecules, including peptidomimetics), or produced recombinantly. In one embodiment, the test agent is a small organic molecule having a molecular weight of less than about 2,000 daltons.

In a further embodiment, the invention provides an assay for identifying a test compound which inhibits or potentiates the activation of a TLR11 and/or TLR12 polypeptide, comprising: (a) forming a reaction mixture including TLR11 or TLR12 polypeptides and a test compound; and (b) detecting activation of said TLR11 or TLR12 polypeptides; wherein a change in the activation of said TLR11 or TLR12 polypeptide in the presence of the test compound, relative to activation in the absence of the test compound, indicates that said test compound potentiates or inhibits activation of said TLR11 and/or TLR12 polypeptide.

Assaying TLR11 or TLR12 complexes, in the presence and absence of a candidate inhibitor, can be accomplished in any vessel suitable for containing the reactants. Examples include microtitre plates, test tubes, and micro-centrifuge tubes.

In one embodiment of the present invention, drug screening assays can be generated which detect inhibitory agents on the basis of their ability to interfere with assembly or stability of a TLR11 or a TLR12 complex. In an exemplary binding assay, the compound of interest is contacted with a mixture comprising a TLR11 or a TLR12 polypeptide and at least one interacting polypeptide. Detection and quantification of TLR11 or TLR12 complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In an additional embodiment of the invention, a TLR11 polypeptide of the invention or fragment thereof is administered to an individual. In another embodiment, a TLR12 polypeptide of the invention or fragment thereof is administered to an individual. In certain embodiments, both a TLR11 polypeptide or fragment thereof and a TLR12 polypeptide or fragment thereof are administered together to an individual. The individual can be a mammal such as a human. When administered to an individual, the TLR11 polypeptide and/or TLR12 polypeptide can be administered as a pharmaceutical composition containing, for example, the TLR11 polypeptide and/or TLR12 polypeptide and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain physiologically acceptable compounds that act, for example, to stabilize or to increase the absorption of the TLR11 polypeptide and/or TLR12 polypeptide. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the composition.

One skilled in the art would know that a pharmaceutical composition containing a TLR11 polypeptide and/or a TLR12 polypeptide can be administered to a subject by various routes including, for example, oral administration; intramuscular administration; intravenous administration; anal administration; vaginal administration; parenteral administration; nasal administration; intraperitoneal administration; subcutaneous administration and topical administration. The composition can be administered by injection or by intubation. The pharmaceutical composition also can be a TLR11 polypeptide and/or TLR12 polypeptide linked to a liposome or other polymer matrix. Liposomes, for example, which consist of phospholipids or other lipids, are nontoxic, physiologically acceptable and metabolizable carriers that are relatively simple to make and administer.

The present invention is illustrated by the following examples, which are not intended to be limiting in any way.

### EXAMPLE 1

### Cloning of mouse TLR 11 and TLR12

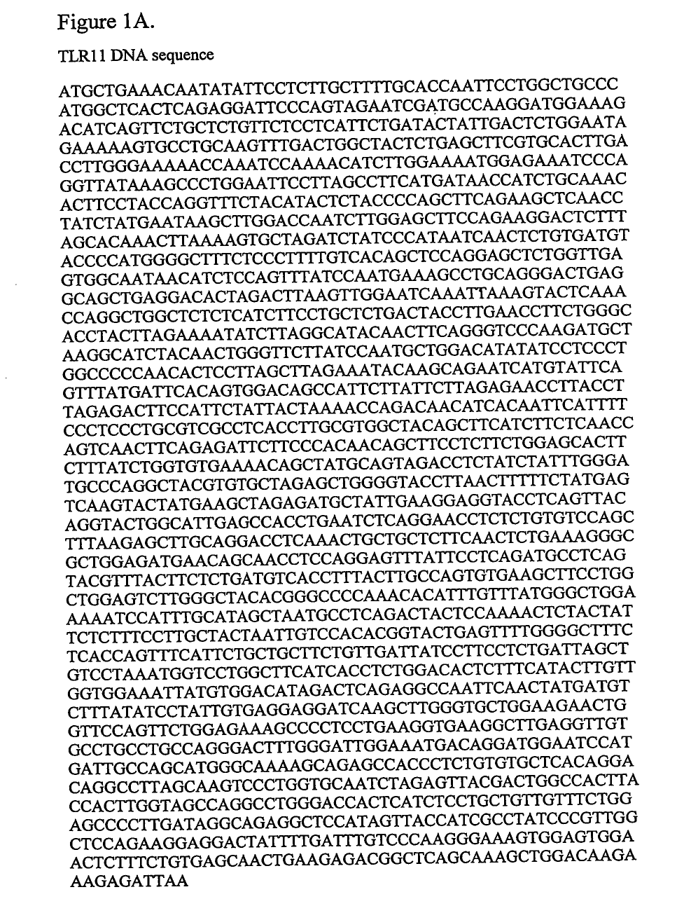

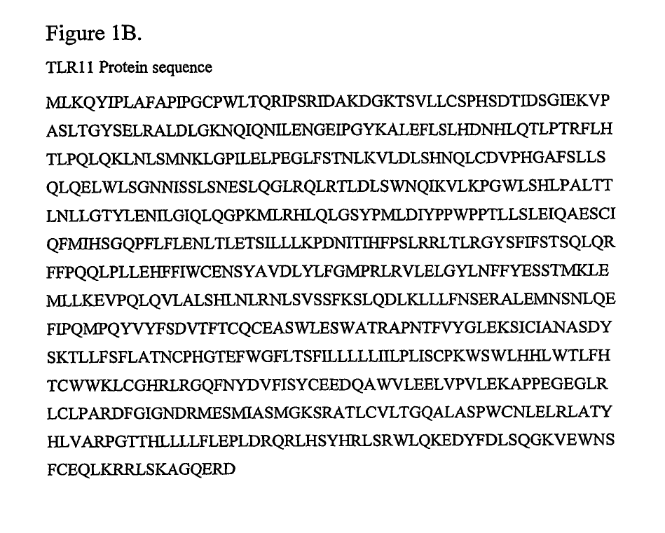

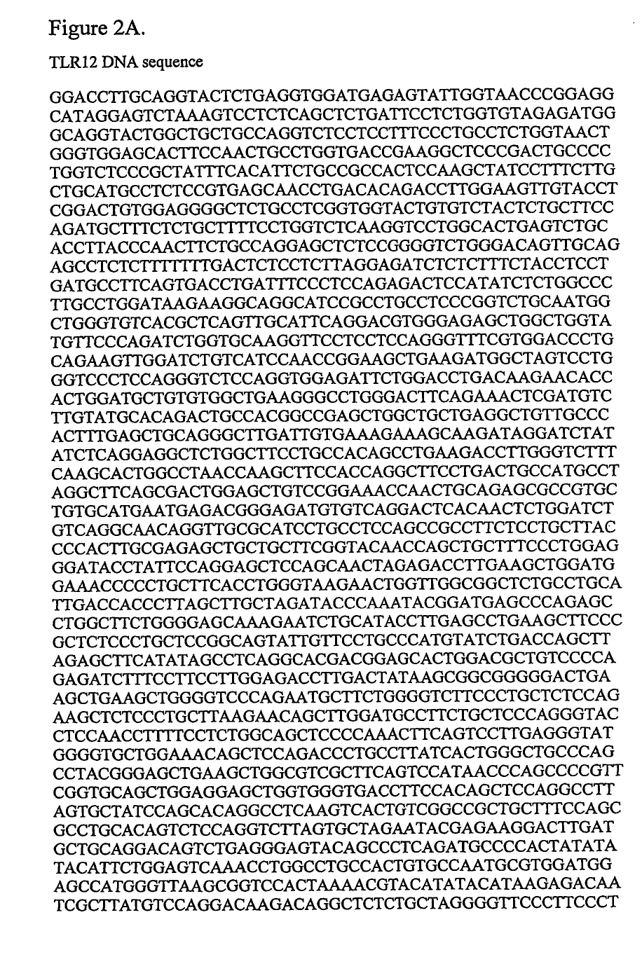

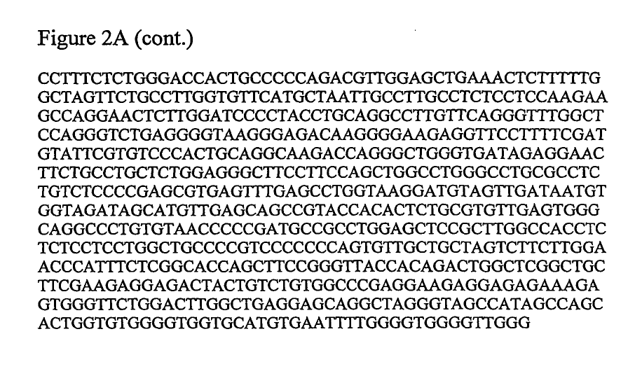

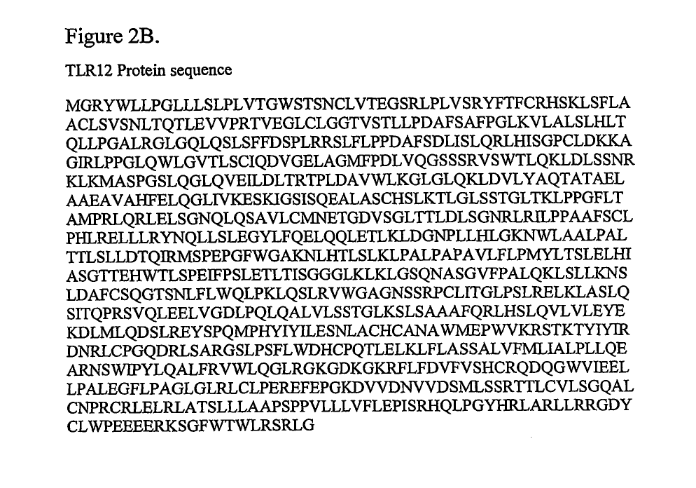

TLR11 and TLR12 were cloned from a mouse splenic cDNA library by reverse transcriptase-polymerase chain reaction (RT-PCR). The primers used to clone TLR11 were 5′ tcaagtctagagccaccatgctgaaacaatatattcctc (forward primer) (SEQ ID NO: 5) and 5′ agtcatctagattaatctctttcttgtccagc (reverse primer) (SEQ ID NO: 6). The primers used to clone TLR12 were 5′ tcaagtctagagccaccatgggcaggtactggctgctgcc (forward primer) (SEQ ID NO: 7) and 5′ aagtcctcgagctaccctagcctgctcctcag (reverse primer) (SEQ ID NO: 8). The nucleic acid sequence of TLR11 is depicted in FIG. 1A, and the amino acid sequence of TLR11 is depicted in FIG. 1B. The nucleic acid sequence of TLR12 is depicted in FIG. 2A, and the amino acid sequence of TLR12 is depicted in FIG. 2B.

### EXAMPLE 2

### TLR11 and TLR12 Induced Activation of Transcription was Measured by Reporter Gene Expression

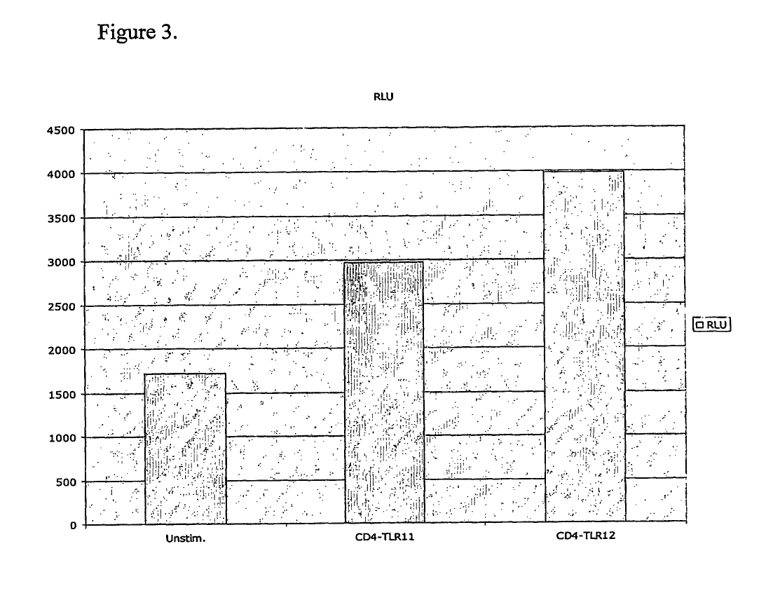

Constitutively active TLR11 and TLR12 were shown to activate NF-κB (FIG. 3). 293T cells were transiently transfected with empty plasmid or with plasmid DNA encoding TLR11 or TLR12 cytoplasmic domains fused to the extracellular portion of CD4 to make them constitutively active along with plasmid encoding an NF-kB dependent luciferase reporter gene using Lipofectamine 2000 (Invitrogen). Twenty-four hours post transfection, cells were harvested by washing once in PBS (potassium phosphate monobasic 0.01M, sodium phosphate dibasic 0.01M, KCl 0.0027M, NaCl 0.137M) and then lysing in TNT lysis buffer (150 mM NaCl, 20 mM Tris pH 8, and Triton X-100, 1%). Lysates were mixed with luciferase substrate (Promega) and luciferase activity was monitored with the use of a luminometer.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An isolated nucleic acid comprising a nucleic acid sequence selected from the group consisting of:
(a) a nucleic acid which is represented by SEQ ID NO: 1;
(b) a nucleic acid which is represented by SEQ ID NO: 3;
(c) a nucleic acid sequence that is at least 70% identical to the nucleic acid sequence of SEQ ID NO: 1;
(d) a nucleic acid sequence that is at least 70% identical to the nucleic acid sequence of SEQ ID NO: 3;
(e) a nucleic acid sequence which is represented by the complement to SEQ ID NO: 1;
(f) a nucleic acid sequence which is represented by the complement to SEQ ID NO: 3;
(g) a nucleic acid sequence that is at least 70% identical to the complement of the nucleic acid sequence represented by SEQ ID NO: 1; and
(h) a nucleic acid sequence that is at least 70% identical to the complement of the nucleic acid sequence represented by SEQ ID NO: 3.

2. An isolated nucleic acid that hybridizes under high stringency conditions to the nucleic acid represented by SEQ ID NO: 1 or SEQ ID NO: 3 or to the complement of SEQ ID NO: 1 or SEQ ID NO: 3.

3. An isolated nucleic acid comprising a nucleic acid sequence that, due to the degeneracy of the genetic code, encodes the amino acid sequence encoded by the nucleic acid sequence depicted in SEQ ID NO: 1 or SEQ ID NO: 3.

4. An isolated Toll-like receptor polypeptide comprising an amino acid sequence selected from the group consisting of:
(a) an amino acid sequence that is at least 70% identical to the amino acid sequence depicted in SEQ ID NO: 2;
(b) an amino acid sequence that is at least 70% identical to the amino acid sequence depicted in SEQ ID NO: 4;
(c) an amino acid sequence that is at least 95% identical to the amino acid sequence depicted in SEQ ID NO: 2;
(d) an amino acid sequence that is at least 95% identical to the amino acid sequence depicted in SEQ ID NO: 4;
(e) an amino acid sequence that is represented by SEQ ID NO: 2; and
(f) an amino acid sequence that is represented by SEQ ID NO: 4.

5. The isolated polypeptide of claim 4, wherein the isolated polypeptide is a variant of a polypeptide represented by SEQ ID NO: 2 or SEQ ID NO: 4.

6. The isolated polypeptide of claim 4, wherein the isolated polypeptide is a fragment of a polypeptide represented by SEQ ID NO: 2 or SEQ ID NO: 4.

7. A vector comprising nucleic acid sequence encoding a polypeptide that is at least 70% identical to the polypeptide represented by SEQ ID NO: 2 or SEQ ID NO: 4.

8. The vector of claim 7, wherein the nucleic acid is operably linked to a transcriptional regulatory sequence.

9. Isolated host cells comprising exogenous nucleic acid encoding a polypeptide that is at least 70% identical to the polypeptide represented by SEQ ID NO: 2 or SEQ ID NO: 4.

10. The isolated host cells of claim 9, wherein the exogenous nucleic acid is a vector.

11. The isolated host cells of claim 10, wherein the vector is a vector of claim 8.

12. A method of producing a Toll-like receptor polypeptide comprising culturing the host cells of claim 11 under conditions suitable for expression of the polypeptide, wherein the Toll-like receptor polypeptide is thereby produced.

13. The method of claim 12, wherein the Toll-like receptor polypeptide is selected from group consisting of polypeptides represented by SEQ ID NO: 2 and SEQ ID NO: 4.

14. A monoclonal or polyclonal antibody, or a chimera or fragment thereof, which is specifically reactive with an epitope of a polypeptide of claim 4.

15. A method for identifying compounds which modulate Toll-like receptor activity comprising:
(a) contacting a polypeptide according to claim 4 with a test agent; and
(b) monitoring for modulation of Toll-like receptor activity,
wherein a compound which modulates Toll-like receptor activity is thereby identified.

16. The method of claim 15, wherein the Toll-like receptor activity monitored in step (b) is NF-κB activation.

17. The method of claim 15, wherein the Toll-like receptor activity is TLR11 activity.

18. The method of claim 15, wherein the Toll-like receptor activity is TLR12 activity.

19. A compound identified by a method according to claim 15.

20. A method of treating an individual having a disorder that is responsive to Toll-like receptor modulation, which method comprises administering to the individual an effective amount of a compound according to claim 19 or an antibody according to claim 14.

21. The method of claim 20, wherein the disorder is selected from the group consisting of: an inflammatory disorder, an autoimmune disease, a cardiovascular disorder, and a systemic infection.

22. The method according to claim 21, wherein the disorder is selected from the group consisting of: a viral, fungal or bacterial infection, including urinary tract infections; asthma; rhinitis; chronic obstructive pulmonary disease (COPD); emphysema; an inflammatory bowel disease such as ulcerative colitis or Crohn's disease; rheumatoid arthritis; osteoarthritis; psoriasis; Alzheimers disease; atherosclerosis, Multiple Sclerosis, diabetes; and septic shock syndrome associated with systemic infection involving gram positive or gram negative bacteria.

23. A polypeptide according to claim 4 for use as an adjuvant.

24. The use of a compound according to claim 19 in the manufacture of a medicament for the treatment of a disorder that is responsive to Toll-like receptor modulation.
